(19) United States
(12) Plant Patent Application Publication
(10) Pub. No.: US 2006/0059595 P1
(43) Pub. Date: Mar. 16, 2006

(54) Verbenaceae hybrid named 'Campagna Verde'

(76) Inventor: Hitoshi Kuramochi, Utsunomiya (JP)

Correspondence Address: Satoshi Yamamoto, 1100 Gough Street #17-F, San Francisco, CA, 94109, US

(21) Appl. No.: 11/257,652

(22) Filed: Oct. 21, 2005

(30) Foreign Application Priority Data
Mar. 15, 2004 (JP) 12041
Dec. 13, 2004 (JP) 17741

(51) Int. Cl. A01H 5/00

(52) U.S. Cl. PLT/263

(57) ABSTRACT

A new cross-species hybrid within the Verbenaceae family, named ‘Campagna-Verde’, having superior vigor and suitability for use as a groundcover, as characterized by fast growth rate, prostrate growth habit forming a dense mat, rare production of seeds, resistance to low and high temperature, resistance to high salinity, resistance to low and high pH extremes, resistance to arid and humid conditions, good tolerance to trampling, and resistance to disease and pests.

### LATIN NAME OF THE GENUS AND SPECIES

Botanical classification: Lippia nodiflora L.

### VARIETY DENOMINATION

The new verbenaceae family groundcover variety denomination is ‘Campagna Verde’.

### BACKGROUND OF THE INVENTION

The present invention comprises a new and distinct cross-species hybrid within the verbena family (Verbenaceae), hereinafter referred to by the name ‘Campagna Verde’.

‘Campagna Verde’ was developed by a controlled crossbreeding, carried out by Hitoshi Kuramochi at Utsunomiya University, Utsunomiya City, Tochigi Prefecture, Japan, between two members of the Verbenaceae family, and selection among the resultant progeny for fast growth to cover areas, good tolerance to low temperature, good tolerance to saline conditions, good tolerance to humid and arid conditions, rare production of seeds, good resistance to being trod on, good tolerance to both high and low pH extremes, and strong resistance to disease and pests. The female parent of ‘Campagna Verde’ used in the cross above is a patented member of the Verbenaceae family, ‘S Hitoshi’ (Japan Patent Registration No. 12041), and the male parent is wild Lippia nodiflora (known as ‘Iwadaresou’) originating from Nansei-Shoto, Southwestern Islands, Okinawa prefecture, Japan. The new hybrid was created at Utsunomiya University, Utsunomiya City, Tochigi Prefecture, Japan, during the period 1999-2004, with crossbreeding carried out in May 1999 and selection over the period May 1999 to May 2002, and has been repeatedly asexually reproduced at Utsunomiya University, Utsunomiya City, Tochigi Prefecture, Japan, using cuttings and layering over a two-year period. It has been found to retain its distinctive characteristics through successive generations of asexual reproduction.

### BRIEF SUMMARY OF THE INVENTION

‘Campagna Verde’ is a groundcover interspecies hybrid of the Verbenaceae family, having superior vigor and suitability for use as a ground cover, as characterized by vigorous prostate growth to cover an area, superior resistance to low temperature, rare production of seeds, and short plants. The data below, which defines the characteristics of ‘Campagna Verde’ and differentiates ‘Campagna Verde’ from its male and female parents, was collected from May 2002 to December 2003 from asexual reproductions carried out at the Utsunomiya University Research Center in Tochigi Prefecture, Japan. The plants were grown both outdoors, in plowed soil supplemented with fertilizer containing 8% N, 8% P, and 8% K, and in temperature-controlled and uncontrolled greenhouses in a mixture of Utsunomiya black soil and river sand, supplemented with the same fertilizer.

### Comparison with Female Parent

Plants of the new verbena family interspecies hybrid ‘Campagna Verde’ are dissimilar to the female parent ‘S-Hitoshi’ in growth rate, resistance to low temperature, frequency of seed production, and plant height. In comparisons done at the Utsunomiya University Research Center (see above), plants of the new hybrid ‘Campagna Verde’ differed from plants of the female parent ‘S-Hitoshi’ in the following characteristics:

1. The new hybrid ‘Campagna Verde’ grows to cover an area two (2) times faster than its female parent ‘S-Hitoshi’.

2. The new hybrid ‘Campagna Verde’ exhibits superior resistance to low temperature extremes compared to its female parent ‘S-Hitoshi’.

3. The new hybrid ‘Campagna Verde’ produces seeds less frequently than its female parent ‘S-Hitoshi’.

4. The new hybrid ‘Campagna Verde’ exhibits plants with height of about 2.0 cm, while its female parent ‘S-Hitoshi’ has plants of about 2.3 cm.

### Comparison with Male Parent

Plants of the new verbena family interspecies hybrid ‘Campagna Verde’ are dissimilar to the male parent, wild Lippia nodiflora (‘Iwadaresou’) originating from the southwestern islands, Okinawa Prefecture, Japan, in growth rate, resistance to low temperature, frequency of seed production, and plant height. They are similar to the male parent Lippia nodiflora ‘Iwadaresou’ in plant and growth habit, as well as leaf form, size, venation, and texture. In comparisons done at the Utsunomiya University Research Center (see above), plants of the new hybrid ‘Campagna Verde’ differed from plants of the male parent Lippia nodiflora ‘Iwadaresou’ in the following characteristics:

1. Plants of the new hybrid ‘Campagna Verde’ grow to cover an area at 3.5 times the rate of plants of its male parent, Lippia nodiflora ‘Iwadaresou’.

2. Plants of the new hybrid ‘Campagna Verde’ exhibit superior resistance to low temperature extremes compared to plants of the male parent Lippia nodiflora ‘Iwadaresou’.

3. Plants of the new hybrid ‘Campagna Verde’ produce seeds less frequently than plants of its male parent Lippia nodiflora ‘Iwadaresou’.

4. Plants of the new hybrid ‘Campagna Verde’ exhibit a height of about 2.0 cm, while plants of its male parent Lippia nodiflora ‘Iwadaresou’ have a height of about 2.5 cm.

In comparisons done at the Utsunomiya University Research Center (see above), plants of the new hybrid ‘Campagna Verde’ were similar to plants of the male parent Lippia nodiflora ‘Iwadaresou’ in the following characteristics:

1. Plants of the new hybrid ‘Campagna Verde’ exhibit a vigorous growing, trailing groundcover form with a procumbent growth habit, forming a dense mat; this is very similar to, or identical to, the form and growth habit of plants of its male parent Lippia nodiflora ‘Iwadaresou’.

2. Plants of the new hybrid ‘Campagna Verde’ have leaves with an obovate shape that is very similar to, or identical to, the shape of leaves of plants of its male parent Lippia nodiflora ‘Iwadaresou’.

3. Plants of the new hybrid ‘Campagna Verde’ have leaves of fairly narrow and fairly short (1.5 cm) dimensions. Leaves of plants of its male parent Lippia nodiflora ‘Iwadaresou’ exhibit very similar dimensions.

4. Plants of the new hybrid ‘Campagna Verde’ have venation very similar to, or identical to, that of plants of its male parent Lippia nodiflora ‘Iwadaresou’.

5. Plants of the new hybrid ‘Campagna Verde’ have hairless leaves of a texture very similar to, or identical to, that plants of its male parent Lippia nodiflora ‘Iwadaresou’.

### BRIEF DESCRIPTION OF ILLUSTRATION

Typical specimens of the plant for the new cross-species hybrid ‘Campagna Verde’ are shown in the accompanying photographs. The colors shown are as true as possible within the usual limits of this kind of illustration.

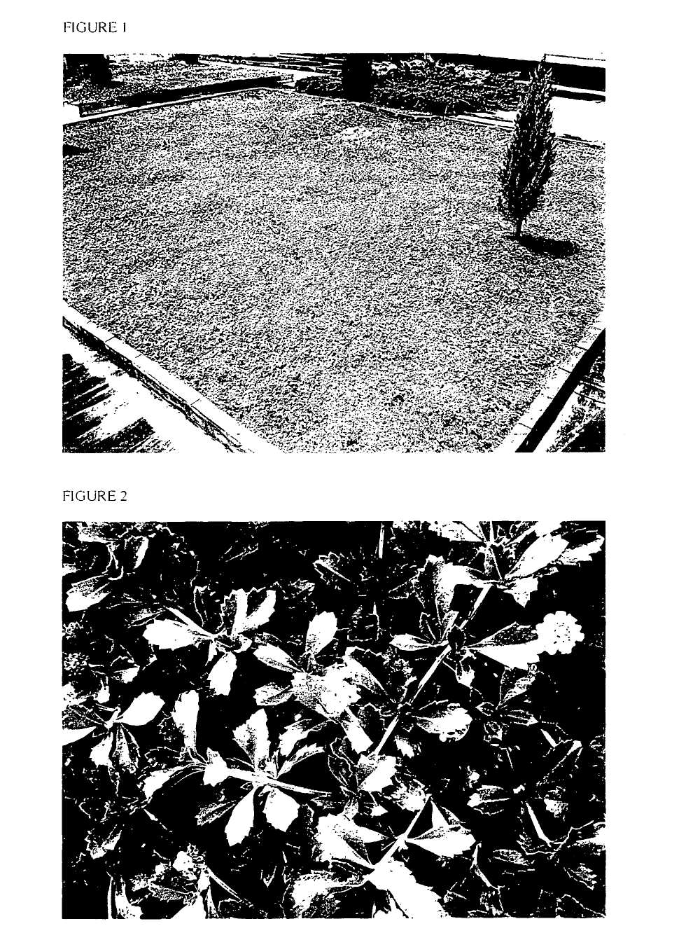

FIG. 1 is a whole plant view of the new hybrid ‘Campagna Verde’, and

FIG. 2 is a side perspective view of a typical flowering stem. The plant shown in the illustration was grown under research conditions at Utsunomiya University, Utsunomiya City, Tochigi Prefecture, Japan.

### DETAILED BOTANICAL DESCRIPTION

The following description of the new verbena family interspecies hybrid ‘Campagna Verde’ is of plants grown from asexual reproductions carried out, with data collected, at Utsunomiya University in Utsunomiya City, Tochigi Prefecture, Japan, in both a research field with plowed soil supplemented with fertilizer containing 8% N, 8% P, and 8% K, and in temperature-controlled and uncontrolled greenhouses in a mixture of Utsunomiya black soil and river sand, supplemented with the same fertilizer, in the time period May 2002-December 2003. The hybrid has not been observed under all possible environmental conditions. The phenotype might vary with variations in the environment such as light intensity without any variance in genotype.

Plants of the new hybrid ‘Campagna Verde’ have shown excellent resistance to high and low temperature extremes, having been grown successfully under temperature conditions ranging from a gentle frost to 37 degrees Celsius. These plants also show, in separate experiments, excellent tolerance to a range of soil pH from 4.0-12.0, humid conditions, arid conditions, high salinity extremes, being trod on or trampled, and excellent resistance to common disease and pests.

The following description is with respect to plants grown under the experimental conditions described above, at Utsunomiya University, Utsunomiya City, Tochigi Prefecture, Japan. The plant and flower measurements and foliage size are established as an average using standard cultural procedures.

### Plant

Height (centimeters): 2.0.
Form: Vigorous growing, trailing groundcover.
Growth habit: Procumbent, forming a dense mat.
Blooming season: late May through mid-November.
Disease and pest resistance: Exhibits very little damage from most common disease and pests; in very humid conditions mildew spotting may appear and can be treated by standard commercially available pesticides.
Drought and temperature resistance: As described above, ‘Campagna Verde’ satisfactorily withstands temperature extremes from a gentle frost to 37 degrees Celsius, and is resistant to humid and arid conditions.
Propagation:
   Type.—Stem cuttings and layering.
   Optimal planting period.—late April to mid-October.
Growth treatments or special conditions: None.

### Foliage

Stem:
   Length.—indefinite.
   Diameter.—Very similar or identical to wild Lippia nodiflora ‘Iwadaresou’.
   Texture.—Very similar or identical to wild Lippia nodiflora ‘Iwadaresou’.
   Color.—Dark green, brownish green.
Arrangement of leaves: Opposite along the stem.
Distance between internodes: 1.6 cm.
Shape of leaf: Obovate, very similar or identical to wild Lippia nodiflora ‘Iwadaresou’.
Size of leaf: Width-fairly narrow, Length (cm): 1.5 cm, very similar or identical to wild Lippia nodiflora ‘Iwadaresou’.
Leaf apex, base, aspect: Very similar or identical to wild Lippia nodiflora ‘Iwadaresou’.
Leaf surface texture.—Hairless, very similar or identical to wild Lippia nodiflora ‘Iwadaresou’.
Leaf venation.—Very similar or identical to wild Lippia nodiflora ‘Iwadaresou’.
Leaf color.—Green, very similar or identical to wild Lippia nodiflora ‘Iwadaresou’.

### Flower

Corolla shape: Conic.
Corolla length: 0.6 cm.
Flower type: Single.
Inflorescence form: Terminal, spikes, on ascending stem of length 1.3 cm.
Diameter of fully expanded blossoms: 0.8 cm.
Number of flowering spikes per plant: Approximately 27.
Fragrance: None.
Color: Purplish-white; JHS No. 8901.
Seeds: Rarely observed.

### Other Distinguishing Characteristics

Resistance to high salinity: Satisfactory.
Growth rate: two (2) times that of female parent, ‘S-Hitoshi’, 3.5 times that of male parent, wild Lippia nodiflora ‘Iwadaresou’.

What is claimed is:

1. A new and distinct cross-species hybrid within the Verbenaceae family, named ‘Campagna-Verde’, substantially as described and illustrated herein.
